(19) United States
(12) Patent Application Publication
(10) Pub. No.: US 2026/0335108 A1
(43) Pub. Date: Oct. 29, 2026

(54) DRAINAGE DEVICE

(71) Applicant: Boston Scientific Scimed, Inc., Maple Grove, MN (US)

(72) Inventor: Martyn G. Folan, Loughrea (IE)

(73) Assignee: Boston Scientific Scimed, Inc., Maple Grove, MN (US)

(21) Appl. No.: 19/662,842

(22) Filed: Apr. 29, 2026

Related U.S. Application Data
(60) Provisional application No. 63/796,474, filed on Apr. 29, 2025.

(51) Int. Cl. A61F 2/86

(52) U.S. Cl. CPC A61F 2/86; A61F 2210/0076; A61F 2230/0069

(57) ABSTRACT

In various aspects, the present disclosure provides a stent that is configured to self-expand from a first constrained configuration to a second unconstrained configuration, the stent in the second unconstrained configuration comprising a tubular duodenal appendage, a first retention member, a tubular saddle region, a second retention member, and a tubular biliary appendage, which together define a lumen that extends through the stent along a longitudinal axis of the stent, wherein the first retention member is positioned along the longitudinal axis between the tubular duodenal appendage and the cylindrical saddle region, wherein the cylindrical saddle region is positioned along the longitudinal axis between the first retention member and the second retention member, and wherein the second retention member is positioned along the longitudinal axis between the cylindrical saddle region and the tubular biliary appendage.

### CROSS-REFERENCE TO RELATED APPLICATIONS

This application claims the benefit of U.S. Provisional Patent Application Serial No. 63/796,474 filed on Apr. 29, 2025, the disclosure of which is incorporated herein by reference.

### FIELD

The present disclosure relates generally to the field of medical devices. In particular, the present disclosure relates to medical devices for facilitating the flow of fluids and materials between adjacent body lumens, such as a stent which maintains an open flow passage between or within body lumens.

### BACKGROUND

Placement of a self-expanding stent (e.g., self-expanding metal stent or SEMS) within an anatomical area (e.g., body lumen, passage, vessel, duct, etc.) may enable fluid communication from one area to another. For example, a stent may enable flow of material from one body lumen to another.

Biliary drainage by endoscopic retrograde cholangiopancreatography (ERCP) in patients with malignant distal bile duct obstruction (MBO) is frequently associated with adverse events, such as pancreatitis, which can hamper patient comfort and outcomes. Endoscopic ultrasound-guided choledochoduodenostomy (EUS-CDS) with a lumen-apposing metal stent (LAMS) is a promising alternative in patients with MBO but is associated with a risk of stent dysfunction, typified by food (chyme) impaction due to the flow of chyme from the gastric region past the lumen-apposing metal stent device. Additionally the angulation of the placed lumen-apposing metal stent may not be conducive to efficient bile flow from the bile duct into the duodenum. Accordingly, a variety of advantageous medical outcomes may be realized by the devices and/or methods of the present disclosure.

### SUMMARY

In various aspects, the present disclosure provides a stent that is configured to self-expand from a first constrained configuration to a second unconstrained configuration, the stent in the second unconstrained configuration comprising a tubular duodenal appendage, a first retention member, a tubular saddle region, a second retention member, and a tubular biliary appendage, which together define a lumen that extends through the stent along a longitudinal axis of the stent, wherein the first retention member is positioned along the longitudinal axis between the tubular duodenal appendage and the cylindrical saddle region, wherein the cylindrical saddle region is positioned along the longitudinal axis between the first retention member and the second retention member, and wherein the second retention member is positioned along the longitudinal axis between the cylindrical saddle region and the tubular biliary appendage.

In some embodiments, a diameter of the tubular saddle region ranges from 6 to 20 mm and a length of the saddle region ranges from 10 to 25 mm.

In some embodiments, which can be used in conjunction with the above aspects and embodiments, a diameter of the tubular duodenal appendage ranges from 6 to 30 mm and a length of the tubular duodenal appendage ranges from 30 to 80 mm.

In some embodiments, which can be used in conjunction with the above aspects and embodiments, a diameter of the tubular biliary appendage ranges from 6 to 20 mm and a length of the tubular biliary appendage ranges from 30 to 80 mm.

In some embodiments, which can be used in conjunction with the above aspects and embodiments, each of the first retention member and the second retention member has a diameter that is greater than a diameter of the cylindrical saddle region, the first retention member has a diameter that is greater than a diameter of the tubular duodenal appendage at an axial location of the tubular duodenal appendage closest to the first retention member, and the second retention member has a diameter that is greater than a diameter of the tubular biliary appendage at an axial location of the tubular biliary appendage closest to the second retention member. In some of these embodiments, the diameter of the first retention member is greater than the diameter of the second retention member. In some of these embodiments, the diameter of the second retention member is greater than the diameter of the first retention member. In some of these embodiments, the diameter of the tubular saddle region is greater than the diameter of the tubular duodenal appendage at the axial location of the tubular duodenal appendage closest to the first retention member.

In some embodiments, which can be used in conjunction with the above aspects and embodiments, the tubular duodenal appendage is tapered such that a diameter of the tubular duodenal appendage decreases with increasing distance from the first retention member.

In some embodiments, which can be used in conjunction with the above aspects and embodiments, the tubular biliary appendage has a flared distal end.

In some embodiments, which can be used in conjunction with the above aspects and embodiments, the first retention member and the second retention member each comprises a double-walled flange having an axially inward wall, an axially outward wall, and a radially outermost region extending therebetween.

In some embodiments, which can be used in conjunction with the above aspects and embodiments, the stent is fully coated with a polymeric layer.

In some embodiments, which can be used in conjunction with the above aspects and embodiments, the tubular duodenal appendage, the first retention member, the first cylindrical saddle region, the second retention member, and the tubular biliary appendage are fully coated with a polymeric layer to prevent leakage therethrough. In some of these embodiments, the stent is fully coated with a polymeric layer except for the axially outward wall of the first retention member. In some of these embodiments, the stent is fully coated with a polymeric layer except for the axially outward wall of the second retention member. In some of these embodiments, the stent is fully coated with a polymeric layer, except for the axially outward wall of the first retention member and the axially outward wall of the second retention member.

In some embodiments, which can be used in conjunction with the above aspects and embodiments, the stent is coated with a polymeric layer such that recessed regions of the double-walled flanges are isolated from a remainder of the lumen that extends through the stent.

In some embodiments, which can be used in conjunction with the above aspects and embodiments, the stent has a unitary construction.

In some embodiments, which can be used in conjunction with the above aspects and embodiments, stent comprises a braid of one or multiple wires.

In various aspects, the present disclosure provides a method of providing a drainage path from a lumen of a biliary duct to a lumen of duodenum in a patient using the stent in accordance with any of the above aspects and embodiments, the method comprising (a) surgically creating a path between the lumen of the duodenum and the lumen of the biliary duct, (b) positioning and expanding the second retention member and the biliary appendage in the lumen of the biliary duct, (c) positioning and expanding the saddle region across walls of the biliary duct and the duodenum, and (d) positioning and expanding the first retention member and the duodenal appendage in the lumen of the duodenum.

The above and other aspects, embodiments and features of the present disclosure will be readily apparent from the following detailed description.

### BRIEF DESCRIPTION OF THE DRAWINGS

Non-limiting examples of the present disclosure are described with reference to the accompanying figures, which are schematic and not intended to be drawn to scale. In the figures, each identical or nearly identical component illustrated is typically represented by a single numeral. For purposes of clarity, not every component is labeled in every figure, nor is every component in each embodiment of the disclosure shown where illustration is not necessary to allow those of skill in the art to understand the disclosure. In the figures:

FIG. 1A illustrates a side view of a medical device according to one or more embodiments described herein.

FIG. 1B schematically illustrates a cross-sectional view of a medical device according to one or more embodiments described herein.

FIG. 1C schematically illustrates a medical device like that of FIG. 1B disposed in tissue according to one or more embodiments described herein.

FIG. 2 illustrates a mandrel for forming a medical device like that of FIG. 1A.

FIG. 3A schematically illustrates a cross-sectional view of a medical device according to one or more embodiments described herein.

FIG. 3B schematically illustrates a cross-sectional view of a medical device according to one or more embodiments described herein.

FIG. 3C schematically illustrates a medical device like that of FIG. 3B disposed in tissue according to one or more embodiments described herein.

FIG. 4 schematically illustrates a cross-sectional view of a medical device according to one or more embodiments described herein.

FIG. 5 schematically illustrates a cross-sectional view of a medical device according to one or more embodiments described herein.

FIG. 6 schematically illustrates a cross-sectional view of a medical device according to one or more embodiments described herein.

FIG. 7A schematically illustrates a cross-sectional view of a medical device according to one or more embodiments described herein.

FIG. 7B schematically illustrates a cross-sectional view of a medical device according to one or more embodiments described herein.

FIG. 7C schematically illustrates a medical device like that of FIG. 7B disposed in tissue according to one or more embodiments described herein.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical devices (e.g., stents) and systems for drainage of the biliary duct, it should be appreciated that such medical devices may be used in a variety of medical procedures (e.g., enteroenterostomy, gastroduodenostomy and gastroileostomy, etc.) to establish and/or maintain a temporary or permanent open flow or drainage passage from or between a variety of body organs, lumens, ducts, vessels, fistulas, cysts and spaces (e.g., the dermis, stomach, duodenum, jejunum, small intestine, gallbladder, kidneys, pancreas, biliary / pancreatic trees, bladder, ureter, abscesses, walled-off pancreatic necrosis (WOPN), bile ducts, etc.). The devices may be inserted via different access points and approaches, e.g., percutaneously, endoscopically, laparoscopically or some combination thereof. The medical devices disclosed herein are self-expanding, but in other embodiments the medical device may be expandable by other means, including, e.g., a balloon catheter. Moreover, such medical devices are not limited to drainage, but may facilitate access to organs, vessels or body lumens for other purposes, such as creating a path to divert or bypass fluids or solids from one location to another, removing obstructions and/or delivering therapy, including non-invasive or minimally invasive manipulation of the tissue within the organ and/or the introduction of pharmacological agents via the open flow passage.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

A medical device, such as a stent, may be formed of one or more filaments and/or surfaces. In various embodiments, one or multiple metal wires, including braids of one or multiple wires, meshes of one or multiple metal wires, and weaves of one or multiple metal wires, one or more polymeric filaments, sheets, or a combination thereof may form a medical device. For example, a length of braid of one or multiple wires may form a medical device. A medical device may include one or more structural elements such as a strut, hoop, mesh, tessellating cell, or other unit. In many embodiments, a medical device may comprise a mesh, weave, and/or knit surface. A medical device may be formed, in various embodiments, of a shape memory material, such as nitinol.

In various embodiments, medical devices described herein are of a unitary construction that is formed, for example, from one or multiple wires or filaments configured as a woven, braided, or knitted construction, or the like.

Various embodiments of medical devices described herein may include a full or partial covering, coating, or other membrane over an interior, over an exterior, extending between structural elements of the devices, or any combination thereof. For example, a covering, coating, or other membrane may comprise a polymer such as a silicone, a polyurethane, a polytetrafluoroethylene, an expanded polytetrafluoroethylene, a polyvinylidene fluoride, an aromatic polycarbonate-based thermoplastic urethane, and/or other like materials. A cover may be applied by dip coating, roll coating, painting, spraying, other known disposition method, or a combination thereof. A covering, coating, or other membrane may inhibit tissue growth and/or minimize fluid leakage from within and/or without the medical device.

In some embodiments, a covering, coating, or other membrane may extend fully or partially over medical devices described herein. For example, a first retention member, a second retention member, a saddle region extending between a first retention member and a second retention member, a duodenal appendage, a biliary appendage, or a combination thereof may comprise a solid covering, a porous covering, or other configuration of covering. In some embodiments, such as stents having a tubular saddle region, a tubular duodenal appendage, a tubular biliary appendage, and double-walled flanges as first and second retention members, a circumferential covering or coating may be applied to cover the full length of the stent, or a partial length of the stent. For example, a partial coating may cover the full length of the saddle region, the duodenal appendage, the biliary appendage, but not all or a portion of the flanges. Embodiments are not limited herein. Specific embodiments are described below.

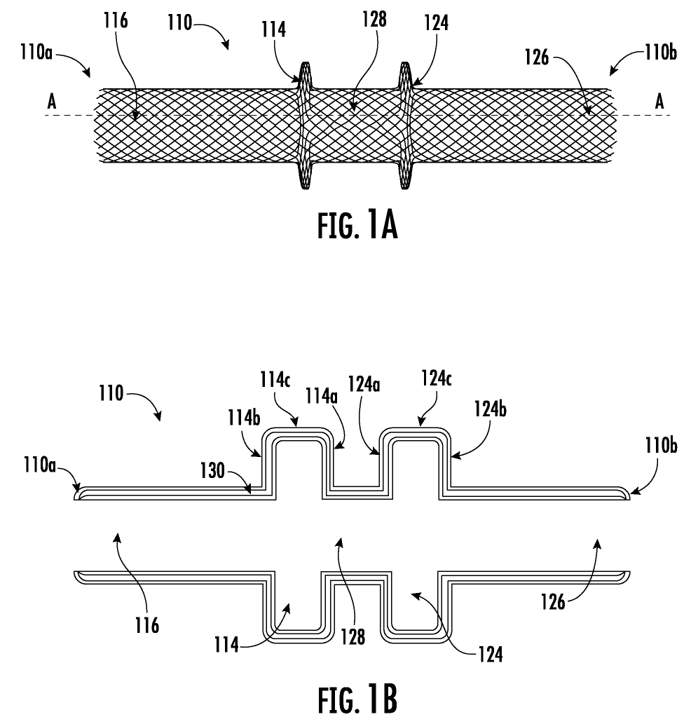

Referring to FIGS. 1A-1B, in one embodiment, a stent 110 of the present disclosure may include a lumen therethrough, an axis A--A, a first end 110a, a duodenal appendage 116, a first retention member 114, a tubular saddle region 128, a second retention member 124, a biliary appendage 126, and a second end 110b. The stent 110 may be covered, uncovered, or a combination thereof. In the embodiment shown, the stent 110 is covered in its entirety.

The stent 110 may include a constrained configuration (e.g., unexpanded or delivery configuration; not shown), and an expanded or unconstrained configuration (e.g., foreshortened or deployed configuration, as shown in FIGS. 1A-1B). The stent 110 may be configured to be expandable between the constrained configuration and the unconstrained configuration. In some embodiments, the stent 110 in the constrained configuration, may comprise a diameter in the range of 2-6 mm, or 3-5 mm. For example, a stent 110 may have a diameter in a constrained configuration of 10Fr or 3.5 mm. In some embodiments, the stent 110 in the constrained configuration may have a length within the range of 40 to 300 mm, more typically, within the range of 60 to 150 mm. In some embodiments, a stent 110 in the constrained configuration may have a length within the range of 80 to 120 mm.

In the unconstrained or expanded configuration shown in FIGS. 1A-1B, the tubular saddle region 128 may extend along a longitudinal axis of the stent 110 between the first retention member 114 and the second retention member 124. The tubular saddle region 128 may comprise a tubular structure or a hollow cylindrical body, defining or otherwise comprising a lumen extending therethrough along the longitudinal axis, or a similar structure. The tubular saddle region 128 may comprise a length and a diameter. In embodiments, such as stents 110 having first and second double-walled flanges as first and second retention members that each comprise an axially inward wall and an axially outward wall, the length of the tubular saddle region may be measured as the length along the stent 110 that is the shortest distance between the first and second flanges at any points along the inward walls of the first and second retention members when the stent 110 is in an unconstrained or expanded configuration. In many embodiments, a tubular saddle region in the unconstrained or expanded configuration may have a length within the range of 5-150 mm, more typically in the range of 8 to 100 mm in the unconstrained or expanded configuration. Exemplary lengths of the tubular saddle region of devices for biliary drainage by endoscopic retrograde cholangiopancreatography may include lengths within a range of 10 to 25 mm.

In various embodiments, a diameter of the tubular saddle region in the unconstrained or expanded configuration may be greater than a diameter of the tubular saddle region in the constrained configuration. For example, a diameter of the tubular saddle region (such as region 128 of FIGS. 1A-1B) in the unconstrained or expanded configuration may be within the range of 3-40 mm. In some embodiments, the diameter of the tubular saddle region 128 in the unconstrained or expanded configuration may be within inclusive ranges of 5 to 25mm. In certain embodiments, the diameter of the tubular saddle region in the unconstrained or expanded configuration may be within inclusive ranges of 6 to 20 mm.

In various embodiments, the diameter of the tubular saddle region 128 in the unconstrained or expanded configuration may be within an inclusive range of 3-5 times greater than a corresponding diameter of the tubular saddle region in the constrained configuration, although in several embodiments, the diameter of the tubular saddle region 128 in the unconstrained or expanded configuration may be 3-10 times greater than the corresponding diameter of the tubular saddle region in the constrained configuration.

In the unconstrained or expanded configuration shown in FIGS. 1A-1B, the duodenal appendage 116 may extend along a longitudinal axis of the stent 110 between the first retention member 114 and the first end 110a of the stent 110. The duodenal appendage 116 may comprise a tubular structure or a cylindrical body, defining or otherwise comprising a lumen extending therethrough along the longitudinal axis, or a similar structure. The duodenal appendage 116 may comprise a diameter and a length. In embodiments, the length of the duodenal appendage 116 may be measured as the length along the duodenal appendage 116 that is the shortest distance between the first end 110a of the stent 110 and the first retention member 114 (e.g., a first flange) at any point along an outward surface of the first retention member (e.g., an outward wall of the first flange) when the stent 110 is in an unconstrained or expanded configuration. In many embodiments, the duodenal appendage 116 may have a length within the range of 10 to 200 mm in the unconstrained or expanded configuration, more typically the duodenal appendage 116 may have a length in the range of 20 to 150 mm in the unconstrained or expanded configuration, even more typically the duodenal appendage 116 may have a length in the range of 30 to 80 mm in the unconstrained or expanded configuration.

In many embodiments, a diameter of the duodenal appendage 116 in the unconstrained or expanded configuration may be greater than a diameter of the duodenal appendage in the constrained configuration. For example, a diameter of the duodenal appendage 116 (such as that of FIGS. 1A-1B) in the unconstrained or expanded configuration may be within the range of 3 to 80 mm. In some embodiments, the diameter of the duodenal appendage 116 in the expanded configuration may be within that range of 6 to 30 mm. In various embodiments, the diameter of the duodenal appendage 116 in the unconstrained or expanded configuration may be 3-5 times greater than a corresponding diameter of the duodenal appendage in the constrained configuration, although in several embodiments, the diameter of the duodenal appendage 116 in the unconstrained or expanded configuration may be 3-10 times greater than the corresponding diameter of the duodenal appendage in the constrained configuration.

In the unconstrained or expanded configuration shown in FIGS. 1A-1B, the biliary appendage 126 may extend along a longitudinal axis of the stent 110 between the second retention member 124 and the second end 110b of the stent 110. The biliary appendage 126 may comprise a tubular structure or a cylindrical body, defining or otherwise comprising a lumen extending therethrough along the longitudinal axis, or a similar structure. The biliary appendage 126 may comprise a diameter and a length. In embodiments, the length of the biliary appendage 126 may be measured as the length along the biliary appendage 126 that is the shortest distance between the second end 110b of the stent 110 and the second retention member 124 (e.g., a second flange) at any point along an outward surface of the second retention member (e.g., an outward wall of the second flange) when the stent 110 is in an unconstrained or expanded configuration. In many embodiments, the biliary appendage 126 may have a length within the range of 10 to 150 mm in the unconstrained or expanded configuration, more typically the biliary appendage 126 may have a length in the range of 20 to 90 mm in the unconstrained or expanded configuration, even more typically the biliary appendage 126 may have a length in the range of 30 to 80 mm in the unconstrained or expanded configuration.

In many embodiments, a diameter of the biliary appendage 126 in the unconstrained or expanded configuration may be greater than a diameter of the biliary appendage in the constrained configuration. For example, a diameter of the biliary appendage 126 (such as that of FIGS. 1A-1B) in the unconstrained or expanded configuration may be within the range of 3 to 30 mm. In some embodiments, the diameter of the biliary appendage 126 in the unconstrained or expanded configuration may be within that range of 6 to 20 mm. In various embodiments, the diameter of the biliary appendage 126 in the unconstrained or expanded configuration may be 3-5 times greater than a corresponding diameter of the biliary appendage in the constrained configuration, although in several embodiments, the diameter of the biliary appendage 126 in the unconstrained or expanded configuration may be 3-10 times greater than the corresponding diameter of the biliary appendage in the constrained configuration.

Referring again to FIG. 1B, the first retention member 114 may extend radially outward from an outer circumference of the saddle region 128 in the unconstrained or expanded configuration to define a double-walled flange with an inward wall surface 114a and an outward wall surface 114b. Similarly, the second retention member 124 may extend radially outward from an outer circumference of the saddle region 128 in the unconstrained or expanded configuration to define a double-walled flange with an inward wall surface 124a and an outward wall surface 124b. For example, inward wall surface 114a may be an axially inward wall of a first flange, outward wall surface 114b may be an axially outward wall of the first flange, inward wall surface 124a may be an axially inward wall of a second flange, and outward wall surface 124b may be an axially outward wall of the second flange. A radially outermost region 114c may extend between and connect inward wall surface 114a and outward wall surface 114b. A radially outermost region 124c may extend between and connect inward wall surface 124a and outward wall surface 124b.

Radially outermost region 114c may comprise a diameter in the unconstrained or expanded configuration that is greater (e.g., a diameter that is 1.5 to 5 times greater) than a diameter of the tubular saddle region 128 at an axial position where the tubular saddle region 128 meets the inward wall surface 114a. Radially outermost region 124c may comprise a diameter in the unconstrained or expanded configuration that is greater (e.g., a diameter that is 1.5 to 5 times greater) than a diameter of the tubular saddle region 128 at an axial position where the tubular saddle region 128 meets the inward wall surface 124a.

Radially outermost region 114c may comprise a diameter in the unconstrained or expanded configuration that is greater (e.g., a diameter that is 1.5 to 3 times greater) than a diameter of the duodenal appendage 116 at an axial position where the duodenal appendage 116 meets the outward wall surface 114b. Radially outermost region 124c may comprise a diameter in the unconstrained or expanded configuration that is greater (e.g., a diameter that is 1.5 to 5 times greater) than a diameter of the biliary appendage 126 at an axial position where the biliary appendage 126 meets the outward wall surface 124b. In various embodiments, a radially outermost region may define a diameter of a respective retention member.

In embodiments, a diameter of the first retention member and/or a second retention member (such as members 114, 124 of FIGS. 1A-1B) in the unconstrained or expanded configuration may be within the range of 5 to 60 mm. Other exemplary retention member diameters in the unconstrained or expanded configuration may be within a range of 10 to 40 mm. In particular embodiments, a diameter of the first retention member and/or a second retention member in the unconstrained or expanded configuration may be within the range of 15 to 35 mm.

In many embodiments, a diameter of a retention member in the unconstrained or expanded configuration may be set to have a particular offset from a diameter of the tubular saddle region in the unconstrained or expanded configuration. For example, a stent 110 may be configured to include a 3-20 mm difference, more typically a 5 to 15 mm difference, between a diameter of the tubular saddle region and a diameter of the first retention member and/or a second retention member in the unconstrained or expanded configuration. In other examples, the first retention member and/or the second retention member may be configured to have a diameter in the unconstrained or expanded configuration that is 1-5 times greater, more typically 1.5 to 5 times greater, than the diameter of the tubular saddle region in the unconstrained or expanded configuration. For example, for a device with the tubular saddle region having a diameter of 10 mm in the unconstrained or expanded configuration, the first retention member and/or a second retention member may have a diameter within inclusive ranges of 15 to 40 mm. It will be understood that some embodiments may include greater or lesser offsets between the diameter of the tubular saddle region and the larger diameter of the first retention member and/or the second retention member.

In many embodiments, a diameter of the first retention member in the unconstrained or expanded configuration may be set to have a particular offset from a diameter of the duodenal appendage in the unconstrained or expanded configuration. For example, a stent 110 may be configured to include a 0 to 50 mm difference, more typically a 10 to 40 mm difference, between a diameter of the first retention member and a diameter of the duodenal appendage where it meets the first retention member. in the unconstrained or expanded configuration. In other examples, the first retention member may be configured to have a diameter in the unconstrained or expanded configuration that is 1-5 times greater, more typically 1 to 3 times greater, than a diameter of the duodenal appendage where it meets the first retention member, in the unconstrained or expanded configuration. It will be understood that some embodiments may include greater or lesser offsets between the diameter of the duodenal appendage and the larger diameter of the first retention member.

In many embodiments, a diameter of the second retention member in the unconstrained or expanded configuration may be set to have a particular offset from a diameter of the biliary appendage in the unconstrained or expanded configuration. For example, a stent 110 may be configured to include a 0 to 30 mm difference, more typically a 5 to 15 mm difference, between a diameter of the second retention member and a diameter of the biliary appendage where it meets the second retention member, in the unconstrained or expanded configuration. In other examples, the second retention member may be configured to have a diameter in the unconstrained or expanded configuration that is 1-5 times greater, more typically 1.5 to 2.5 times greater, than a diameter of the biliary appendage where it meets the second retention member, in the unconstrained or expanded configuration. It will be understood that some embodiments may include greater or lesser offsets between the diameter of the biliary appendage and the larger diameter of the second retention member.

Referring again to FIGS. 1A-1B, the first retention member 114 may have a same or different axial width as the second retention member 124, wherein the axial width of a retention member may be measured as a distance along the longitudinal axis between an inward wall and outward wall of a respective retention member, inclusive of the radially outermost region. In some embodiments, the width of the first retention member 114 and/or the second retention member 124 in the expanded configuration may be within the inclusive range of 0.5-10.0 mm. Other embodiments may include a smaller and/or greater width of the first retention member and/or the second retention member, such as within the inclusive rages of 0.5-6 mm, 2-6 mm, or 3-7 mm. In some embodiments, a retention member may have a constant width. In other embodiments, a width of a retention member may vary along a vertical plane.

Various embodiments may include total stent 110 lengths ranging from 40 to 140 mm in the unconstrained or expanded configuration, more typically, 60 to 100 mm.

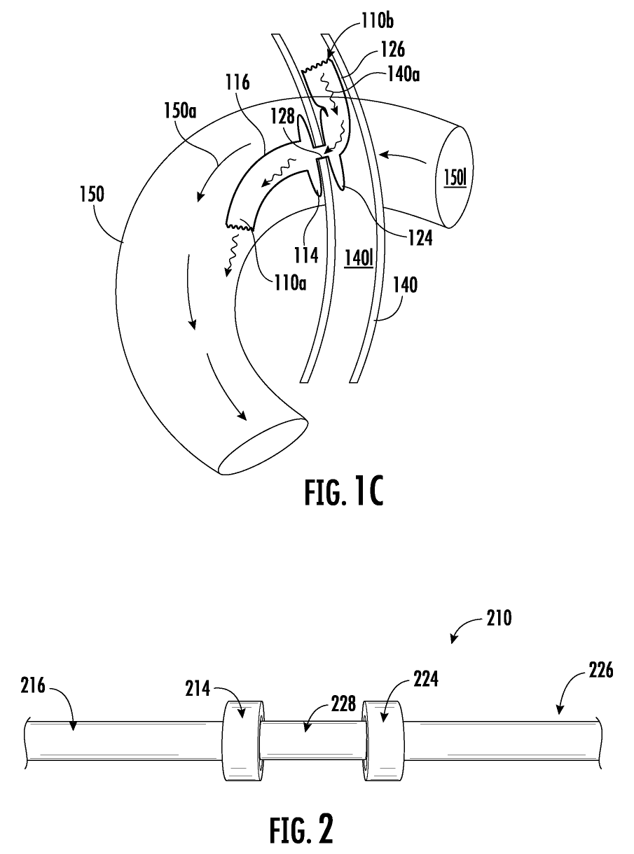

A medical device 110 like that shown in FIGS. 1A-1B may be formed around a mandrel 210 that includes a central mandrel providing a saddle-forming region 228, a duodenal appendage-forming region 216, and a biliary appendage-forming region 216, as well as a first flange-forming washer 214 and a second flange-forming washer 224. For example, a length of a braid of one or multiple wires may be deposited or formed on the mandrel 210. The shape of the braided wire may be set, for example, in an expanded configuration (e.g., unconstrained configuration) of a self-expanding body. For example, the braided wire may comprise a shape-memory material, such as Nitinol or a similar alloy. The shape may be set, for example, by heat-setting. After the shape of the stent structure has been set, the central mandrel, first flange-forming washer and second flange-forming washer may be removed. The medical device 110 can then be coated with a suitable coating material 130. For example, the medical device can then 110 be coated while positioned on a mandrel having a non-adherent surface and a shape like that of FIG. 2 through a suitable coating process such as a spray coating, dip coating, roll coating, hand painting, spraying, other known coating method, or a combination thereof. Further description of suitable techniques for shaping and coating medical devices in accordance with the present disclosure can be found in U.S. Patent Publication Nos. US20220203077A1 and US20220079784A1, the disclosures of which are hereby incorporated by reference.

A medical device 110 like that shown in FIGS. 1A-1B may be implanted in a subject in a single procedure that includes (a) surgically creating a path (e.g., a cauterized path) between the duodenum and the biliary duct, (b) positioning and expanding the second retention member (e.g., biliary flange) and biliary appendage in the biliary tract, (c) positioning and expanding the saddle region across the biliary duct and duodenal walls, (d) positioning and expanding the first retention member (e.g., duodenal flange) and duodenal appendage in the duodenal lumen.

In some embodiments, the medical device is implanted along the lines described in U.S. Patent No. 11,850,172 B2, the disclosure of which is hereby incorporated by reference. In this method, the medical device may be disposed in a constrained configuration between an inner member and an outer sheath of a tissue-penetrating element. For example, in a constrained configuration, one or more of the duodenal appendage 116, first retention member 114, a tubular saddle region 128, second retention member 124 and biliary appendage 126 may be constricted to a smaller diameter, as compared to those features in an expanded or unconstrained configuration. A sharpened end of the tissue penetrating element may be included to advance through a first tissue layer from a first body lumen (e.g., through a duodenal wall from a duodenal lumen). The sharpened end of the tissue penetrating element may be advanced through a second tissue layer and into a second body lumen (e.g., through a biliary duct wall into a biliary duct lumen). Additionally, or alternatively, the tissue penetrating element may comprise an electrically conductive tip for advancing through the first tissue layer and/or the second tissue layer. In various embodiments, a gap space may exist between the first tissue layer and the second tissue layer prior to the deployment of the medical device. With the end of the tissue penetrating element advanced through the second tissue layer, the outer sheath may be proximally retracted with respect to the inner member, or the inner member may be distally extended with respect to the outer sheath, thereby allowing a distal end of the medical device to move from the constrained configuration to an unconstrained configuration. In particular, the distal end of the medical device may expand to deploy the second retention member 124 and the biliary appendage 126. The medical device may be positioned such that a portion of the inward wall surface 124a of the second retention member 124 contacts the second tissue layer. The tissue penetrating element may be proximally retracted through the second tissue layer so that the second retention member 124 and the biliary appendage 126 remain in the second body lumen. The tissue penetrating element may be further proximally retracted through the first tissue layer.

After deploying the second retention member 124 and the biliary appendage 126, the medical device may be retracted proximally a distance, e.g., a predetermined distance that may be indicated by a visual (such as colored band) and/or imageable (such as radiopaque band) marker(s) on the device, along with the entire assembly of inner member and outer sheath, so that the first retention member remains constrained between the two and is not prematurely deployed (e.g. in any gap space between the first and second tissue layers). Once the second retention member 124 is deployed, the action of proximally retracting the entire assembly may also act to draw the first and second tissue layers into apposition or further into apposition with each other. When the device is retracted a sufficient distance to clear the first tissue layer, the outer sheath may be proximally retracted with respect to the inner member, or the inner member may be distally extended with respect to the outer sheath, thereby allowing a proximal end of the medical device to move from the constrained configuration to an unconstrained configuration. In particular, the proximal end of the medical device may expand to deploy saddle region 128, the first retention member 114 and the duodenal appendage 116.

Alternatively, in the methods above, a separate instrument with a sharpened tip may be advanced along the path above and into the second body lumen to create a path, a guidewire put in place, and the separate instrument withdrawn over the guidewire. A stent according to the various embodiments described above may be loaded on a delivery catheter and inserted over the guidewire, and the stent 110 then deployed according to the steps outlined above.

FIG. 1C is a schematic illustration of a fully delivered stent of the present disclosure. For example, the stent of FIG. 1C may be a stent as described with respect to FIGS. 1A-1B. Various components of the stent may be similar to components described above. The stent may be positioned within a patient such that the inward wall surface of the first retention member 114 contacts an inner surface of a duodenal wall 150 (not illustrated) and the inward wall surface of the second retention member 124 contacts an inner surface of a biliary duct wall 140, with the tubular saddle region 128 extending therebetween. In some embodiments, the first retention member 114 and the second retention member 124 may contact the duodenal wall 150 and the biliary duct wall 140, respectively, so that the duodenal wall 150 and the biliary duct wall 140 interact at a tissue interface. In some embodiments, the first retention member 114 and the second retention member 124 may interact with the duodenal wall 150 and the biliary duct wall 140 to cause an interaction of the respective tissue walls at an interface so as to decrease or eliminate a width of a space therebetween. A lumen extending through the duodenal appendage 116, first retention member 114, a tubular saddle region 128, second retention member 124, and biliary appendage 126 may therefore provide an open interior passage between the biliary duct lumen 140l and the duodenal lumen 150l. Flow of food (chyme) within the duodenal lumen 150l is illustrated by arrows 150a. Flow of bile within the biliary duct lumen 140l and stent is illustrated by arrows 140a. The stent may be partially or fully covered, for example, to prevent fluid draining from the biliary duct lumen 140l to the duodenal lumen 150l from leaking through the retention members and/or saddle region of the stent to within any gap space between the duodenal wall 150 and the biliary duct wall 140. Additionally, due to the presence of the elongate biliary appendage 126 and its positioning in the bile duct upstream of the surgically created path between the biliary duct and duodenum, the angulation of the placed stent is conducive to efficient bile flow from the biliary duct 140 into the duodenum 150.

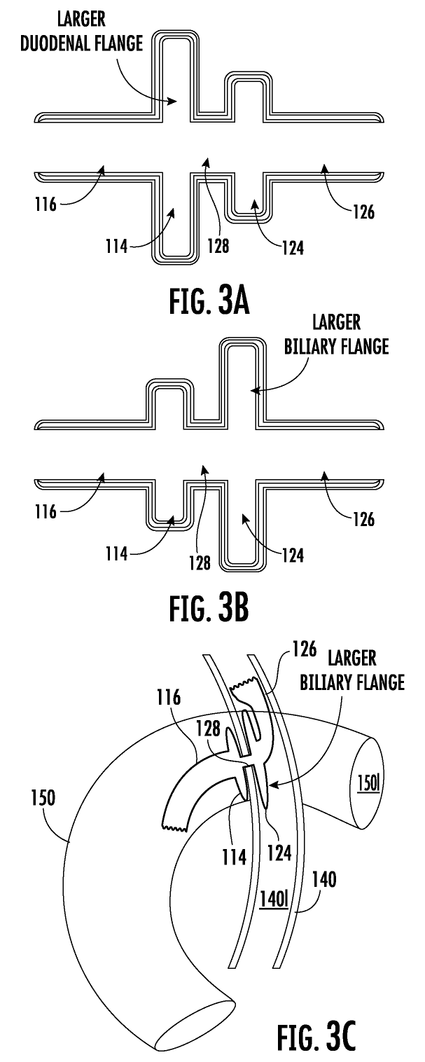

Another embodiment of a stent in accordance with the present disclosure is shown in FIG. 3A, which, like FIG. 1B, includes a lumen therethrough, a duodenal appendage 116, a first retention member (e.g., a duodenal flange 114), a tubular saddle region 128, a second retention member (e.g., a biliary flange 124) and a biliary appendage 126. In FIG. 3A, however, the duodenal flange 114 has a diameter that is greater than a diameter of the biliary flange 124. For example, the duodenal flange 114 may have a diameter that is 1 to 4 times greater, typically 1.5 to 3 times greater, than a diameter of the biliary flange 124.

A further embodiment of a stent in accordance with the present disclosure is shown in FIG. 3B, which, like FIG. 1B, includes a lumen therethrough, a duodenal appendage 116, a first retention member (e.g., a duodenal flange 114), a tubular saddle region 128, a second retention member (e.g., a biliary flange 124) and a biliary appendage 126. In FIG. 3B, however, the biliary flange 124 has a diameter that is greater than a diameter of the duodenal flange 114. For example, the biliary flange 124 may have a diameter that is 1 to 4 times greater, typically 1.5 to 3 times greater, than a diameter of the duodenal flange 114.

In FIGS. 3A and 3B, the larger flange may be placed on the duodenal side (FIG. 3A) or biliary side (FIG. 3B) depending on which direction increased anti-migration resistance is desired. The designs may be achieved by the use of a mandrel with appropriately sized flange-forming washers prior to annealing.

FIG. 3C is a schematic illustration of a fully delivered stent as described with respect to FIG. 3B. The stent is positioned such that the inward wall surface of the first retention member 114 contacts an inner surface of a duodenal wall 150 and the inward wall surface of the second retention member 124 contacts an inner surface of a biliary duct wall 140, with the tubular saddle region 128 extending therebetween. A lumen extending through the duodenal appendage 116, first retention member 114, a tubular saddle region 128, second retention member 124 and biliary appendage 126 may therefore provide an open interior passage between the biliary duct lumen 140l and the duodenal lumen 150l. As in FIG. 1C, due to the presence of the elongate biliary appendage 126, the angulation of the placed stent is conducive to efficient bile flow from the biliary duct 140 into the duodenum 150.

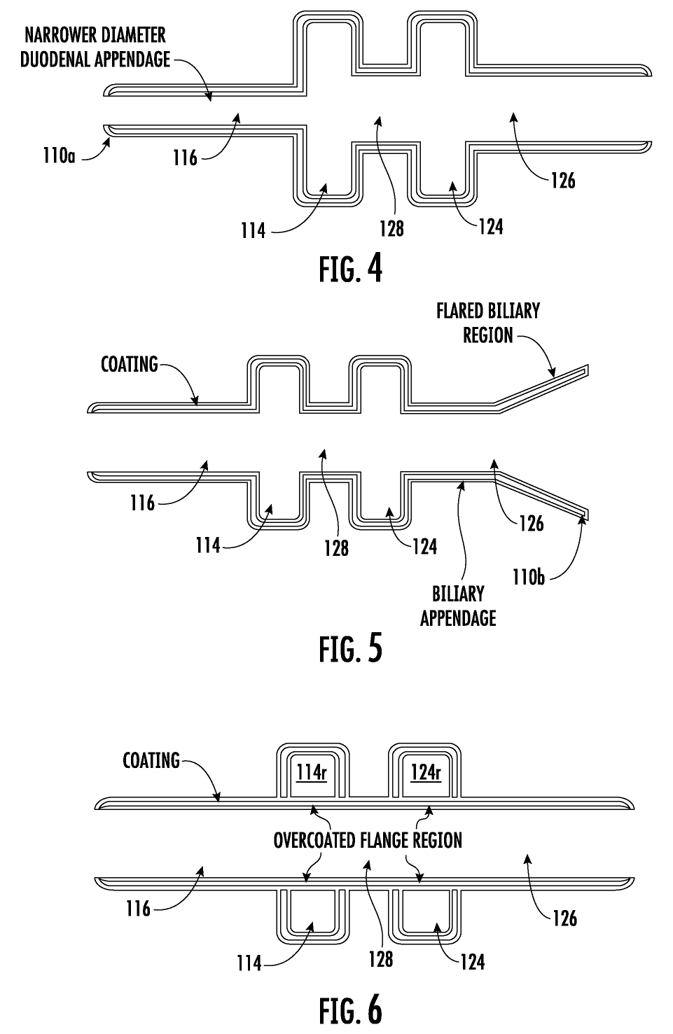

Yet another embodiment of a stent in accordance with the present disclosure is shown in FIG. 4, which, like FIG. 1B, includes a lumen therethrough, a duodenal appendage 116, a first retention member (e.g., a duodenal flange 114), a tubular saddle region 128, a second retention member (e.g., a biliary flange 124) and a biliary appendage 126. In FIG. 4, however, the duodenal appendage 116 has a diameter that is smaller than a diameter of the biliary appendage 126. For example, the biliary appendage 126 may have a diameter that is 1 to 3 times, typically 1.5 to 2 times the diameter of the duodenal appendage 116. In other embodiments, the duodenal appendage 116 may be tapered such that a diameter of the duodenal appendage 116 where it transitions to the duodenal flange 114 is 1 to 3 times, typically 1.5 to 2 times, a diameter of the duodenal appendage 116 at the first end of the device 110a. The use of a duodenal appendage having a tapered or narrower diameter allows for bile flow to be directed towards the duodenal outlet while reducing the likelihood of food impaction.

Another embodiment of a stent in accordance with the present disclosure is shown in FIG. 5, which, like FIG. 1B, includes a lumen therethrough, a duodenal appendage 116, a first retention member (e.g., a duodenal flange 114), a tubular saddle region 128, a second retention member (e.g., a biliary flange 124) and a biliary appendage 126. In FIG. 5, however, the biliary appendage 126 has a diameter that flares outward at the second end of the device 110b. For example, the biliary appendage 126 may have a diameter at the second end of the device 110b is 1 to 3 times, typically 1.5 to 2 times, a diameter of the biliary appendage 126 where it transitions to the biliary flange 124. The use of a biliary appendage that is flared may be more adaptable for different biliary diameters. In contrast, a biliary appendage of a fixed diameter, if placed into a duct of larger diameter, may lead to less effective drainage through the device. A flared biliary appendage may better fit the duct size and improve this drainage through the device.

An additional embodiment of a stent in accordance with the present disclosure is shown in FIG. 6, which, like FIG. 1B, includes a lumen therethrough, a duodenal appendage 116, a first retention member (e.g., a duodenal flange 114), a tubular saddle region 128, a second retention member (e.g., a biliary flange 124) and a biliary appendage 126. Like FIG. 1B, a coating is provided that covers the duodenal appendage 116, duodenal flange 114, tubular saddle region 128, biliary flange 124 and biliary appendage 126. However, in FIG. 6, the stent is coated such that the coating is continuous through both appendage sections and the saddle region and is formed to occlude the recessed double wall flange sections. This can be achieved by coating the stent while it is loaded onto a cylindrical mandrel having a non-adherent surface leading to the formation of a cylindrical coating layer that extends the length of the device. In a subsequent step the outer walls of the duodenal flange 114 and biliary flange 124 may be separately coated (e.g., by hand painting the flanges 114, 124) to form a fully coated device as illustrated in FIG. 6. This allows for the bile flow to be directed through the device without the possibility of bile build up within the recessed regions 114r, 124r of the flanges 114, 124.

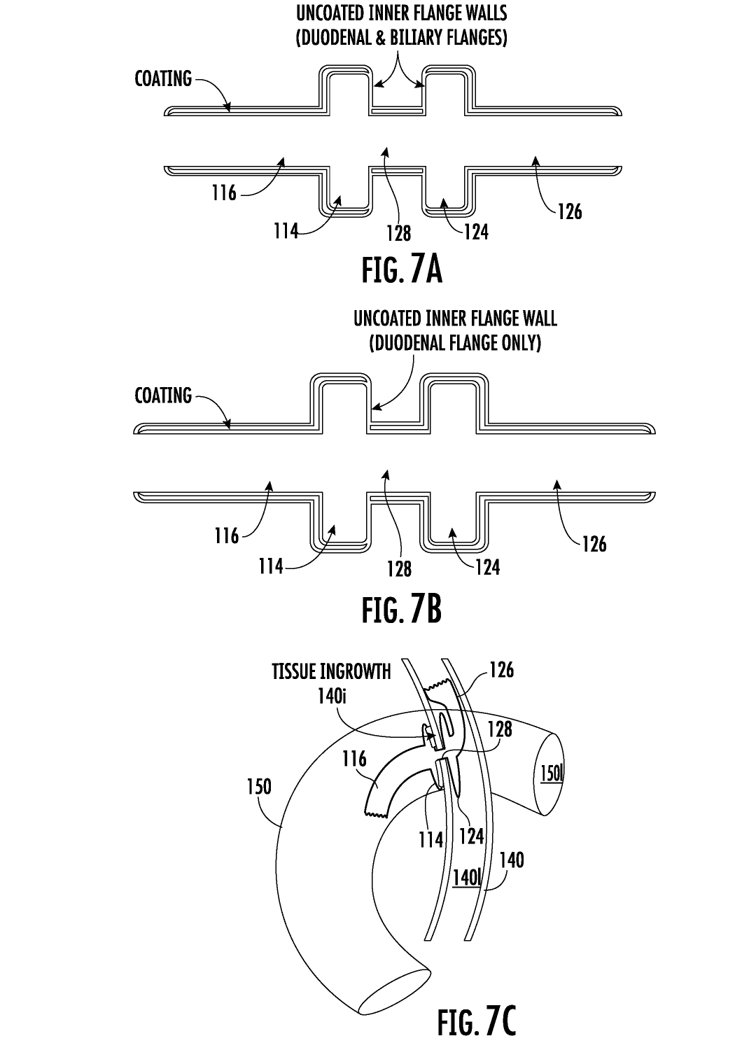

Further embodiments of stents in accordance with the present disclosure are shown in FIGS. 7A-7B, which, like FIG. 1B, include a lumen therethrough, a duodenal appendage 116, a first retention member (e.g., a duodenal flange 114), a tubular saddle region 128, a second retention member (e.g., a biliary flange 124) and a biliary appendage 126. A coating is provided in FIG. 7A that covers the duodenal appendage 116, duodenal flange 114 (except for the inner wall), tubular saddle region 128, biliary flange 124 (except for the inner wall) and biliary appendage 126. A coating is provided in FIG. 7B that covers the duodenal appendage 116, duodenal flange 114 (except for the inner wall), tubular saddle region 128, biliary flange 124 and biliary appendage 126.

By selectively leaving bare the inner walls of the double wall flanges, when placed in the body, this allows for tissue ingrowth into the bare flange face, potentially improving anti-migration of the device. The use of a short saddle region can aid in direct contact between these bare regions and the duct walls, ensuring ingrowth. Various alternatives of this concept are envisaged with combinations of uncoated flange faces. The design of FIG. 7A allows for tissue ingrowth of duodenal tissue into the duodenal flange 114 and for tissue ingrowth of duodenal tissue into the biliary flange 124. The design of FIG. 7B allows for tissue ingrowth of duodenal tissue into the duodenal flange 114.

FIG. 7C is a schematic illustration of a fully delivered stent as described with respect to FIG. 7B. The stent is positioned such that the inward wall surface of the duodenal flange 114 contacts an inner surface of a duodenal wall 150 and the inward wall surface of the biliary flange 124 contacts an inner surface of a biliary duct wall 140, with the tubular saddle region 128 extending therebetween. A lumen extending through the duodenal appendage 116, first retention member 114, tubular saddle region 128, second retention member 124 and biliary appendage 126 may therefore provide an open interior passage between the biliary duct lumen 140l and the duodenal lumen 150l. The design of FIG. 7B allows for tissue ingrowth of duodenal tissue 140i into the duodenal flange 114.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1. A stent configured to self-expand from a first constrained configuration to a second unconstrained configuration, the stent in the second unconstrained configuration comprising a tubular duodenal appendage, a first retention member, a tubular saddle region, a second retention member, and a tubular biliary appendage, which together define a lumen that extends through the stent along a longitudinal axis of the stent, wherein the first retention member is positioned along the longitudinal axis between the tubular duodenal appendage and the cylindrical saddle region, wherein the cylindrical saddle region is positioned along the longitudinal axis between the first retention member and the second retention member, and wherein the second retention member is positioned along the longitudinal axis between the cylindrical saddle region and the tubular biliary appendage.

2. The stent of claim 1, wherein a diameter of the tubular saddle region ranges from 6 to 20 mm and wherein a length of the saddle region ranges from 10 to 25 mm.

3. The stent of claim 1, wherein a diameter of the tubular duodenal appendage ranges from 6 to 30 mm and wherein a length of the tubular duodenal appendage ranges from 30 to 80 mm.

4. The stent of claim 1, wherein a diameter of the tubular biliary appendage ranges from 6 to 20 mm and wherein a length of the tubular biliary appendage ranges from 30 to 80 mm.

5. The stent of claim 1, wherein each of the first retention member and the second retention member has a diameter that is greater than a diameter of the cylindrical saddle region, wherein the first retention member has a diameter that is greater than a diameter of the tubular duodenal appendage at an axial location of the tubular duodenal appendage closest to the first retention member, and wherein the second retention member has a diameter that is greater than a diameter of the tubular biliary appendage at an axial location of the tubular biliary appendage closest to the second retention member.

6. The stent of claim 5, wherein the diameter of the first retention member is greater than the diameter of the second retention member.

7. The stent of claim 5, wherein the diameter of the second retention member is greater than the diameter of the first retention member.

8. The stent of claim 5, wherein the diameter of the tubular saddle region is greater than the diameter of the tubular duodenal appendage at the axial location of the tubular duodenal appendage closest to the first retention member.

9. The stent of claim 1, wherein the tubular duodenal appendage is tapered such that a diameter of the tubular duodenal appendage decreases with increasing distance from the first retention member.

10. The stent of claim 1, wherein the tubular biliary appendage has a flared distal end.

11. The stent of claim 1, wherein the first retention member and the second retention member each comprises a double-walled flange having an axially inward wall, an axially outward wall, and a radially outermost region extending therebetween.

12. The stent of claim 1, wherein the stent fully coated with a polymeric layer.

13. The stent of claim 1, wherein the tubular duodenal appendage, the first retention member, the first cylindrical saddle region, the second retention member, and the tubular biliary appendage are fully coated with a polymeric layer to prevent leakage therethrough.

14. The stent of claim 11, wherein the stent is fully coated with a polymeric layer except for the axially outward wall of the first retention member or except for the axially outward wall of the second retention member.

15. The stent of claim 11, wherein the stent is fully coated with a polymeric layer except for the axially outward wall of the first retention member and the axially outward wall of the second retention member.

16. The stent of claim 11, wherein the stent is coated with a polymeric layer such that recessed regions of the double-walled flanges are isolated from a remainder of the lumen that extends through the stent.

17. The stent of claim 1, wherein the stent has a unitary construction.

18. The stent of claim 1, wherein stent comprises a braid of one or multiple wires.

19. A stent configured to self-expand from a first constrained configuration to a second unconstrained configuration, the stent in the second unconstrained configuration comprising a tubular duodenal appendage, a first retention member, a tubular saddle region, a second retention member, and a tubular biliary appendage, which together define a lumen that extends through the stent along a longitudinal axis of the stent, wherein the first retention member is positioned along the longitudinal axis between the tubular duodenal appendage and the cylindrical saddle region, wherein the cylindrical saddle region is positioned along the longitudinal axis between the first retention member and the second retention member, wherein the second retention member is positioned along the longitudinal axis between the cylindrical saddle region and the tubular biliary appendage, wherein a diameter of the tubular saddle region ranges from 6 to 20 mm, wherein a length of the saddle region ranges from 10 to 25 mm, wherein a diameter of the tubular duodenal appendage ranges from 6 to 30 mm, wherein a length of the tubular duodenal appendage ranges from 30 to 80 mm, wherein a diameter of the tubular biliary appendage ranges from 6 to 20 mm, and wherein a length of the tubular biliary appendage ranges from 30 to 80 mm.

20. A method of providing a drainage path from a lumen of a biliary duct to a lumen of duodenum in a patient using a stent that is configured to self-expand from a first constrained configuration to a second unconstrained configuration, the stent in the second unconstrained configuration comprising a tubular duodenal appendage, a first retention member, a tubular saddle region, a second retention member, and a tubular biliary appendage, which together define a lumen that extends through the stent along a longitudinal axis of the stent, wherein the first retention member is positioned along the longitudinal axis between the tubular duodenal appendage and
the cylindrical saddle region, wherein the cylindrical saddle region is positioned along the longitudinal axis between the first retention member and the second retention member, and wherein the second retention member is positioned along the longitudinal axis between the cylindrical saddle region and the tubular biliary appendage, the method comprising: (a) surgically creating a path between the lumen of the duodenum and the lumen of the biliary duct, (b) positioning and expanding the second retention member and the biliary appendage in the lumen of the biliary duct, (c) positioning and expanding the saddle region across walls of the biliary duct and the duodenum, and (d) positioning and expanding the first retention member and the duodenal appendage in the lumen of the duodenum.
